(19) United States
(12) Patent Application Publication
(10) Pub. No.: US 2010/0036180 A1
(43) Pub. Date: Feb. 11, 2010

(54) METHOD OF OBTAINING 1,2-DICHLOROETHANE BY DIRECT CHLORINATION WITH A STEP OF SEPARATION FROM THE CATALYST BY DIRECT EVAPORATION, AND FACILITY FOR THE IMPLEMENTATION THEREOF

(76) Inventors: Philippe Leduc, Larajasse (FR); Francois Vanney, Lyon (FR); Remy Teissier, Francheville (FR)

Correspondence Address: ARKEMA INC.;PATENT DEPARTMENT - 26TH FLOOR, 2000 MARKET STREET, PHILADELPHIA, PA, 19103-3222, US

(21) Appl. No.: 12/373,388

(22) PCT Filed: Jul. 10, 2007
(86) PCT No.: PCT/FR07/51629

§ 371 Date: Sep. 16, 2009

(30) Foreign Application Priority Data
Jul. 13, 2006 (FR) 0652951

(51) Int. Cl. C07C 21/04; B01J 10/00

(52) U.S. Cl. 570/220; 422/189

(57) ABSTRACT

The present invention relates to a method of producing liquid 1,2-dichloroethane (DCE), obtained by low-temperature direct chlorination of ethylene, in the presence of a Lewis acid-type catalyst, that makes it possible to obtain, after separation of the catalyst, DCE of sufficient purity to give, via cracking, vinyl chloride monomer (VCM); characterized in that it comprises a step of dechlorination (5) of the liquid DCE stream (4) exiting the chlorination reactor (1), that makes it possible to remove the excess dissolved chlorine, followed by a step of direct evaporation (9) of the whole of the liquid DCE stream (8) exiting said reactor, that makes it possible to separate the catalyst from the evaporated fraction (10) of the stream of DCE good for cracking.

The invention also relates to the plant for the implementation of such a method.

The present invention relates to a novel method for producing liquid 1,2-dichloroethane (subsequently referred to as DCE), obtained by low-temperature direct chlorination of ethylene by chlorine, in the presence of a Lewis acid-type catalyst, that makes it possible to obtain by direct evaporation, after separation of the catalyst, DCE of pure quality for cracking (thermal cracking) to vinyl chloride monomer (VCM). The invention also relates to a plant for the implementation thereof.

The direct chlorination reaction of ethylene in the liquid phase is the following:

C₂H_{4(g)}+Cl_{2(g)}→C₂H₄CL_{2(liq)}(DCE)(exothermic reaction,ΔH=−220 kJ/mol) (1)

The thermal cracking of DCE to obtain VCM takes place according to the following reaction:

C₂H₄Cl₂→C₂H₃Cl(VCM)+HCl (2)

Another reaction known as oxychlorination makes it possible to enhance the value of the HCl produced and to obtain DCE according to (3):

C₂H₄+2HCl+½O₂→C₂H₄Cl₂+H₂O (3)

The two main industrial methods for producing DCE, well known in the prior art, that are currently used are:
the method by low-temperature direct chlorination (at a temperature less than or equal to 80° C.) starting from ethylene and chlorine, and under a pressure of 1 to 2 bar, in particular in a loop reactor, in the presence of a catalyst based on FeCl₃, formed in situ; the reaction takes place in the liquid DCE in the presence of dissolved Cl₂. After dechlorination with sodium hydroxide and washing with water to remove the catalyst therefrom, the crude DCE is distilled in several columns to attain the required purity (>99.5%) for cracking; and
the method by high-temperature chlorination (temperature greater than 80° C.), starting from ethylene and chlorine, and under a pressure such that the DCE produced may be directly recovered in the gas phase (free of catalyst) either by boiling, or by expansion; however, the DCE obtained under these conditions generally requires additional distillation steps to attain the pure quality for cracking.

These methods are, in particular, described in the following documents.

Document DE 33 47 153 describes a method for producing DCE by low-temperature direct chlorination starting from ethylene and chlorine, in the presence of a catalyst based on FeCl₃ and an amine, in which the product obtained passes into a distillation column in order to obtain DCE having a purity of 99.9%, one portion from the bottom of the column containing the catalyst being recycled to the reactor. The distillation step is not avoided.

Document WO 96/03361 or EP 772 576 describes a method and a device for producing DCE by direct chlorination starting from ethylene and chlorine, in the presence of a catalyst based on FeCl₃ and on NaCl; the main stream of DCE exiting the reactor is recycled toward the latter, whereas one portion of the DCE is vaporized by expansion, the vapor portion being free of catalyst and having, after condensation and recovery of its heat vaporization, a purity of at least 99.9%, whereas the liquid portion of DCE (at the expansion valve) is recycled to the reactor. The exemplary embodiment indicates a chlorination temperature of 90° C.; this is therefore not a low-temperature direct chlorination.

Document WO 01/21564 or EP 1 214 279 describes a method for recovering the heat during the production of DCE by high-temperature direct chlorination starting from ethylene and chlorine; the DCE vapors exiting the reactor are compressed and are used to feed evaporators of DCE drying and/or distillation columns or heat exchangers. This is therefore not low-temperature direct chlorination.

Moreover, document EP 0 795 531 in the name of the Applicant describes a method for converting light by-products that have a boiling point very close to that of DCE (83.7° C. at atmospheric pressure) formed during the thermal cracking of DCE, in which the chlorination of said light by-products is carried out directly after the direct chlorination reactor, in the presence of the products from this reactor, at a temperature between 20° C. and 80° C., with molecular chlorine. This is not low-temperature direct chlorination.

Document DE 199 16 753 or EP 1 044 950 describes a method for producing DCE by direct chlorination at a temperature between 75 and 125° C., starting from ethylene and chlorine, with recovery of the heat from the chlorination reaction, in order to heat columns for distilling the DCE originating from the oxychlorination and from the cracking. No indication is given on the treatment of the DCE obtained by direct chlorination and it is not a low-temperature direct chlorination.

One of the major problems encountered in the method for producing 1,2-dichloroethane (DCE), obtained by low-temperature direct chlorination, namely the separation on the one hand of DCE of pure quality for cracking, and on the other hand, of the catalyst, is only solved in the various documents cited, by using several steps, on the one hand, washing the crude DCE with water to eliminate the catalyst (FeCl₃) therefrom, and on the other hand of distilling the wet DCE, expensive both in terms of plant and thermal energy.

Another problem is that it is necessary to work in an excess of chlorine to attain a good productivity (from 500 to 1500 ppm of Cl₂ dissolved in DCE) and at a low level of energy, which prevents the use of any method for obtaining DCE good for cracking by simple expansion of the mixture exiting the reactor.

Surprisingly, the Applicant has found a satisfactory solution to these problems by combining steps of dechlorination and direct evaporation requiring a supply of energy, for the stream of crude DCE that enables the separation of the catalyst and of the DCE that is pure (or good) for cracking.

The dechlorination step allows the removal of the excess chlorine dissolved in the DCE stream exiting the direct chlorination reactor.

The steps of evaporation then of condensation may be carried out by the use of systems that enable energy savings, such as the mechanical compression of the vapors or multiple-effect evaporation, with considerable reductions in vapor consumption.

Another advantage of this method is that the catalyst thus separated may be recycled to the reactor for direct chlorination of ethylene by chlorine, then operating with a lesser excess of chlorine, which leads to less corrosion of the reactor, an improvement in the quality of the crude DCE exiting the latter and also an improvement in the productivity.

Another advantage is that a purge of recycled DCE containing the catalyst may also be used to improve the chlorination of the light by-products formed during the thermal cracking of the DCE.

This method finally has the advantage of being able to be integrated into a project for improving or increasing the capacity of an existing plant, by freeing capacity in the distillation line in place, in a relatively simple manner, and by decreasing the aqueous effluents from the washing of the crude DCE.

One subject of the present invention is a method for producing liquid 1,2-dichloroethane (DCE), obtained by low-temperature direct chlorination of ethylene, in the presence of a Lewis acid-type catalyst, that makes it possible to obtain, after separation of the catalyst, DCE of sufficient purity to give, via cracking, vinyl chloride monomer (VCM); characterized in that it comprises a step of dechlorination of the liquid DCE stream exiting the chlorination reactor, that makes it possible to remove the excess dissolved chlorine, followed by a step of direct evaporation of the whole of the liquid DCE stream exiting the reactor, that makes it possible to separate the catalyst from the evaporated fraction of the stream of DCE good for cracking.

According to the invention, the dechlorination step that enables the elimination of the excess chlorine dissolved in the liquid DCE stream exiting the direct chlorination reactor is carried out either by chemical reaction by introducing ethylene into this liquid DCE stream, or by stripping with an inert gas.

During the step of evaporating the liquid DCE stream, a fraction of the liquid DCE remains in contact with the catalyst as evaporation bottoms, so as to be completely or partly recycled to the direct chlorination reactor.

According to the invention, in the evaporation step, the liquid DCE is brought to a vaporization temperature between 75° C. (under a pressure of 0.77 bar, i.e. 0.077 MPa) and 120° C. (under a pressure of 2.8 bar, i.e. 0.28 MPa), and preferably at a temperature of around 84° C. under a pressure of 1 bar (0.1 MPa).

According to a first preferred variant of the invention, following the evaporation step, the DCE vapors undergo a mechanical compression, preferably at a pressure ranging from 1.1 to 2.8 bar (i.e. 0.11 to 0.28 MPa), and more particularly at around 1.6 bar (0.16 MPa), and a condensation at a temperature between 85 and 120° C., and more particularly at around 106° C., enabling the condensation energy to be recovered. This energy may advantageously be used for the vaporization of DCE.

According to another embodiment variant, the evaporation and condensation steps of the DCE are carried out, in particular, by multiple-effect type heat exchangers.

In one embodiment variant according to the invention, the evaporation and condensation steps are followed by a step of secondary purification of the DCE. In particular, this step of secondary purification of the DCE allows the separation of the light compounds such as ethylene and ethyl chloride, which may have a harmful effect, depending on the cracking conditions, for the thermal cracking of the liquid fraction of purified DCE that is good for cracking.

Preferably, one portion of the liquid fraction of DCE from the evaporation bottoms enriched with catalyst (known as purge) is used for the chlorination of the light by-products obtained in the DCE cracking step.

Among these light by-products, mention will especially be made of unsaturated aliphatic hydrocarbons such as benzene, chloroprene or trichloroethylene. These products being difficult to separate from DCE by distillation.

Preferably, the Lewis acid-type catalyst is based on ferric chloride (FeCl₃).

Another subject of the present invention is a plant for implementing the method described previously, which comprises, after a low-temperature direct chlorination reactor (1), fed with chlorine (2) and ethylene (3), a tank (5) for dechlorination via introduction of ethylene (6) into the crude liquid DCE stream (4) exiting the reactor, followed by an evaporation device (9), the inlet (8) of which is fed by the whole of said dechlorinated liquid DCE stream exiting said reactor (1), of which the outlet (11) corresponds to the liquid DCE, concentrated in catalyst, which is completely or partly recycled (12) to the reactor (1), and of which the outlet (10) corresponds to the vaporized DCE good for cracking.

In particular, the evaporation device (9) is composed of any device comprising a heat exchanger that supplies the energy necessary for vaporization of the DCE.

According to a first preferred embodiment, the DCE vapors exiting at (10) are mechanically compressed and condensed in a device (15) that comprises a compressor operating at a discharge pressure between 1.1 and 2.8 bar (i.e. 0.11 to 0.28 MPa), and in particular of around 1.6 bar (0.16 MPa).

According to a second preferred embodiment, the evaporation device (9) and condensation device (15) are composed of a series of multiple-effect type heat exchangers.

Advantageously, the stream of liquid DCE and of gas (18) exiting the condensation device (15) undergoes a treatment in a secondary purification device (19), comprising in particular at least one column for distillation or for stripping with inert gases, to remove the gases (21) such as ethylene, hydrogen chloride and ethyl chloride and to supply even purer DCE (20) for cracking.

Furthermore, one portion (13) of the liquid DCE concentrated in catalyst (11) from the evaporation device (9) is introduced into a reactor (14) for chlorination of the light by-products (17) from the step of cracking DCE to VCM, with supply of chlorine (16), the products (22) of which, after washing and distillation, make it possible to recover pure DCE.

In the first embodiment according to the invention, with mechanical compression of the DCE vapors, the gains obtained as savings in vapor due to the fact that the DCE originating from the direct chlorination no longer passes through the traditional distillation columns are much greater than the electricity consumption due to the compressor.

For a low-temperature direct chlorination unit producing 50 t/h of DCE, the comparative energy balance between the conventional distillation washing method and the method according to the invention brings out a saving of at least 13 t/h of vapor.

### DCE Quality Obtained:

DCE: 99.91% by weight
EtCl (ethyl chloride): 25 ppm by weight;
T112 (1,1,2-trichloroethane): 800 ppm by weight.

### INDUSTRIAL EXEMPLARY EMBODIMENT

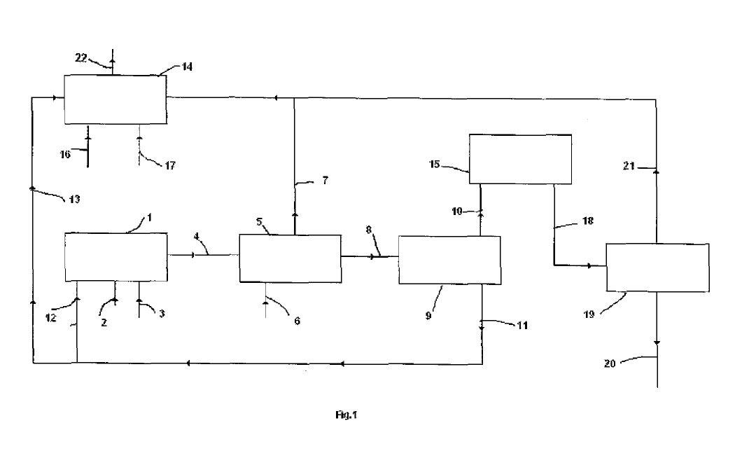

This example is described while referring to FIG. 1 below, which schematically illustrates the method and the device according to one preferred embodiment of the invention.

The production of 1,2-dichloroethane (DCE) is carried out in a loop reactor (1), by low-temperature direct chlorination at T=62.4° C. and P=1.3 bar (0.13 MPa) starting from ethylene (2), flow rate of 7242 kg/h and excess chlorine (3), flow rate of 18 590 kg/h, in the presence of a catalyst based on FeCl₃, in an amount of 170 ppm. The stream (4), flow rate of 59 862 kg/h, exiting said reactor (1) comprises crude DCE, as a mixture with FeCl₃, chlorine, ethyl chloride, and 1,1,2-trichloroethane (T112).

This stream (4) is then conveyed to a dechlorination tank (5) with the introduction of ethylene (6), flow rate of 64 kg/h, one portion of unconsumed ethylene being extracted at (7) with DCE at the vapor pressure, flow rate of 10 kg/h, and recycled toward a unit (14) for chlorination of the “light” by-products, which will be explained in detail below.

The stream (8) exiting this dechlorination tank (5), which still contains the same by-products as a mixture with the DCE, except the chlorine, is introduced into an evaporation device (9), of which the vapor phase outlet of DCE (10), flow rate of 52 711 kg/h, T=84° C., P=1 bar (0.1 MPa), is introduced into a condensation device (15), and of which the liquid phase outlet (11) of the DCE, concentrated in catalyst, flow rate of 12 177 kg/h, is partly recycled (12), flow rate of 10 177 kg/h, to the direct chlorination reactor (1).

At the outlet (18) of the condensation device (15) the flow rates of DCE, C₂H₄, EtCl and T112 are respectively 48 635, 44, 8 and 39 kg/h; the whole mixture is then conveyed to a secondary purification device (19) comprising in particular a column for distillation or stripping with inert gases, in order to remove the gases (21) such as ethylene, the flow rate of which is 44 kg/h, and ethyl chloride, flow rate of 7 kg/h, and to provide pure DCE (20) for cracking, at a flow rate of 47 593 kg/h.

The unit (14) for chlorination of the “light” by-products is fed by one portion known as a purge (13) originating from the DCE evaporation device (9), flow rate of 1994 kg/h, containing FeCl₃, 840 ppm, and T112, flow rate of 4 kg/h, and also by Cl₂ (16), flow rate of 200 kg/h, and light compounds (17), flow rate of 3000 kg/h, derived from the step of cracking the DCE to VCM, after passing through a distilling column; the products (22) exiting this unit, after washing and distillation, make it possible to recover DCE that is good for cracking.

Example of the Laboratory Operation of the Chlorination with Direct Evaporation

Introduced continuously over a bottoms of liquid DCE in a mini-chlorinator made of glass and having a volume of 300 cc equipped with a specimen of iron, are chlorine at a fixed flow rate of 10 l/h, ethylene at a flow rate controlled at around 10-11 l/h, air at a flow rate of 1 l/h and nitrogen at 9 l/h. The flow rate of ethylene is controlled so as to stabilize the chlorine content in the reactor at a chosen value.

The continuous chlorination is carried out at 60° C.

The DCE produced is recovered by overflowing, then is dechlorinated by stripping with nitrogen and finally it is conveyed to a heated evaporator: the evaporated then recondensed DCE represents the production, the bottoms from the evaporator are either stored or sent to the chlorinator.

The test is carried out in two stages:

First stage: duration of 110 h without recycling of the bottoms from the evaporator to the chlorinator; chlorine dissolved in the chlorinator=1000 ppm; ethylene in the vents=1% (volume); and FeCl₃ in the chlorinator between 85 and 115 ppm.

Second stage: duration of 392 h with recycling of the bottoms from the evaporator to the chlorinator.

The content of FeCl₃ in the chlorinator gradually increases to reach 380 ppm at the end of the test. The effect of the iron content is substantial from the start of the recycling: to maintain the content of 1% ethylene in the vents, it is necessary to work with 600 ppm of chlorine dissolved in the chlorinator.

Table 1 below presents the composition of the chlorinator during the test and the purity of the DCE produced by the method (determined by gas phase chromatography: GPC, expressed in % by weight), with the content of T112, expressed as % by weight. The purity of the DCE is stable and corresponds to that of the DCE good for cracking.

TABLE 1

| | | GPC | | | GPC | |
|---|---|---|---|---|---|---|
| Details | Hours | purity | % T112 | Details | purity | % T112 |
| DCE | 120 | 99.89 | 0.063 | DCE | 99.98 | 0.021 |
| reactor | 144 | 99.90 | 0.057 | production | 99.96 | 0.032 |
| | 168 | 99.90 | 0.052 | | 99.96 | 0.030 |
| | 192 | 99.86 | 0.067 | | 99.94 | 0.048 |
| | 264 | 99.85 | 0.059 | | 99.96 | 0.033 |
| | 312 | 99.80 | 0.067 | | 99.95 | 0.039 |
| | 336 | 99.77 | 0.08 | | 99.94 | 0.044 |
| | 360 | 99.76 | 0.080 | | 99.94 | 0.049 |
| | 384 | 99.76 | 0.078 | | 99.94 | 0.046 |
| | 432 | 99.66 | 0.092 | | 99.92 | 0.056 |
| | 480 | 99.76 | 0.062 | | 99.95 | 0.033 |
| | 503 | 99.73 | 0.069 | | 99.95 | 0.034 |

1. A method for producing liquid 1,2-dichloroethane (DCE), via low-temperature direct chlorination of ethylene, in the presence of a Lewis acid-type catalyst, characterized in that it comprises a step of dechlorination of a liquid DCE stream exiting a chlorination reactor, followed by a step of direct evaporation of the whole of the liquid DCE stream exiting said reactor, whereby the catalyst is separated from the evaporated fraction of the stream of DCE.

2. The method as claimed in claim 1, characterized in that the step of dechlorination of the liquid DCE stream exiting the chlorination reactor is carried out by introducing ethylene into the liquid DCE stream, or by stripping with an inert gas.

3. The method as claimed in claim 1, characterized in that during the step of evaporation of the whole of the liquid DCE stream, a fraction of the liquid DCE evaporation bottoms is enriched with a catalyst as and recycled to the chlorination reactor.

4. The method as claimed in claim 1, characterized in that in the direct evaporation step, the liquid DCE is brought to a vaporization temperature between 75° C. (under a pressure of 0.77 bar, i.e. 0.077 MPa) and 120° C. (under a pressure of 2.8 bar, i.e. 0.28 MPa.

5. The method as claimed in claim 1, characterized in that following the evaporation step, the DCE vapors undergo a mechanical compression, at a pressure ranging from 1.1 to 2.8 bar (i.e. 0.11 to 0.28 MPa), and a condensation at a temperature between 85 and 120° C.

6. The method as claimed in claim 5, characterized in that the evaporation and condensation steps are carried out, by multiple-effect type heat exchangers.

7. The method as claimed in claim 1, characterized in that it further comprises, a step of purifying of the DCE, by distillation or stripping.

8. The method as claimed in claim 3, characterized in that a portion of the DCE evaporation bottoms enriched with catalyst used for the chlorination of light by-products obtained in a DCE cracking step.

9. A plant for implementing the method as claimed in claim 1, characterized in that it comprises, a low-temperature direct chlorination reactor (1), fed with chlorine (2) and ethylene (3), thereafter a dechlorination tank (5) feed with ethylene (6) and a crude DCE stream (4) from the chlorination reactor, followed by an evaporation device (9), an inlet of which is fed by the whole of said dechlorinated DCE stream (8) exiting said chlorination reactor (1), the outlet (11) of which is a of liquid-phase DCE, concentrated in catalyst, which is completely or partly recycled (12) to the chlorination reactor (1), the outlet (10) of which corresponds to the stream of vaporized DCE.

10. The plant as claimed in claim 9, characterized in that the evaporation device (9) is composed of any device comprising a heat exchanger that supplies the energy necessary for vaporization of the DCE.

11. The plant as claimed in claim 9, characterized in that DCE vapors (10) exiting the evaporation device (9) are mechanically compressed and condensed in a device (15) that comprises a compressor operating at a discharge pressure between 1.1 and 2.8 bar (i.e. 0.11 to 0.28 MPa).

12. The plant as claimed in claim 9, characterized in that the evaporation device (9) and condensation device (15) comprise a series of multiple-effect type exchangers.

13. The plant as claimed in claim 11, characterized in that a liquid DCE stream (18) exiting the condensation device (15) undergoes a treatment in a secondary purification device (19), comprising a column for distillation or for stripping with inert gases.

14. The plant as claimed in claim 9, characterized in that one portion (13) of the liquid phase DCE concentrated in catalyst (11) from the evaporation device (9) is introduced into a reactor (14) for chlorination of the light by-products (17) from a step of cracking DCE to VCM, the gaseous products (22) of which, are washed and distilled to recover-DCE.

15. The method of claim 4 wherein said temperature is about 84° C. under a pressure of 1 bar (0.1 MPa).

16. The method of claim 5 wherein said pressure is about 1.6 bar (0.16 MPa).

17. The method of claim 5 wherein said temperature is about 106° C.

18. The plant as claimed in claim 11 wherein said, discharge pressure is around 1.6 bar (0.16 MPa).
